(19) United States
(12) Patent Application Publication
(10) Pub. No.: US 2014/0203176 A1
(43) Pub. Date: Jul. 24, 2014

(54) SYSTEMS AND METHODS FOR REAL-TIME SAMPLING AND ANALYSIS OF BIOMOLECULES BENEATH THE SURFACE OF BIOLOGICAL TISSUE

(71) Applicant: DOW AGROSCIENCES LLC, INDIANAPOLIS, IN (US)

(72) Inventors: SURESH BABU ANNANGUDI PALANI, Zionsville, IN (US); Jeffrey R. Gilbert, Zionsville, IN (US)

(73) Assignee: DOW AGROSCIENCES LLC, INDIANAPOLIS, IN (US)

(21) Appl. No.: 14/030,628

(22) Filed: Sep. 18, 2013

Related U.S. Application Data
(60) Provisional application No. 61/755,523, filed on Jan. 23, 2013.

(51) Int. Cl. G01N 33/00; H01J 49/26

(52) U.S. Cl. CPC G01N 33/0098; H01J 49/26
USPC 250/282; 250/288

(57) ABSTRACT

Provided are systems and methods for real-time sampling and analysis of biomolecules beneath the surface of biological/agricultural tissue/sample. A method for determining fatty acid profiles in agricultural products (for example, seeds) comprises using matrix-assisted laser desorption ionization (MALDI) mass spectroscopy or laser ablation electrospray ionization (LAESI) mass spectroscopy. The MALDI or LAESI mass spectroscopy may be used to profile certain fatty acid traits, such as docosahexanoic acid (DHA), in oil seeds. The disclosed method may be used for a high throughput and/or automated screening of agricultural products, such as seeds, for desirable traits or events.

### CROSS-REFERENCE TO RELATED APPLICATION

This application claims the benefit of U.S. Provisional Patent Application Ser. No. 61/755,523, filed Jan. 23, 2013, the disclosure of which is hereby incorporated herein in its entirety by this reference.

### FIELD OF THE INVENTION

This invention is generally related to the field of seed sampling, and more specifically the field of mass spectrometric analysis and laser ablation.

### BACKGROUND OF THE INVENTION

Certain fatty acids in seeds, such as docosahexanoic acid (DHA, omega-3 fatty acid), continue to gain an increased market appeal due to their health values. Determination of the fatty acid profiles of a population of seeds is laborious and time consuming. The commonly used methods involve sampling seeds and extracting the components of interest from the seed sample for analysis. These methods result in destruction of seeds. Non-destructive analysis of seeds is important because it allows for the selection of events with desired traits while keeping the seeds viable.

Screening of fatty acid content in oil seeds is typically performed using fatty acid methyl ester (FAME) analysis, wherein fatty acid in oil seeds is converted to fatty ester and gas chromatography is used to analyze the generated fatty ester. FAME analysis is a destructive technique, which prevents the seeds from being reused. For example, U.S. Patent Publication No. 2007/0048872 discloses a method for determining fatty acid profiles in seeds using FAME analysis. The method involves extracting oil from the seed sample, transesterifying the extracted oil to form a mixture of fatty acid esters, and analyzing the mixture of fatty acid esters using a gas chromatography with flame ionization detection (GC/FID) to determine the fatty acid profiles of the seed sample.

U.S. Pat. No. 6,809,819 discloses a method for determining oil content in seeds using evaporative light scattering detection technique. The method involves extracting oil from a seed sample using a solvent, evaporating the solvent in a stream of gas to form oil particles, directing light into the stream of gas and the oil particles to cause a reflected light from the oil particles, and determining the oil content based on the reflected light.

U.S. Patent Publication No. 2012/0092663 discloses a method of analyzing seeds or grains using transmission Raman spectroscopy (TRS) to determine the composition of the seeds, such as protein and oil content.

Quantitative analysis of oil or fatty acid content in seeds is often performed using conventional methods, such as near infrared analysis (NIR), nuclear magnetic resonance imaging (NMR), soxhlet extraction, accelerated solvent extraction (ASE), microwave extraction, and super critical fluid extraction. These methods are time consuming and not amenable to high-throughput screening of seeds.

Thus, there remains a need for seed sampling technology to characterize oil and/or protein content from agricultural samples including seeds, and such seed sampling technology is adaptable for high-throughput and/or automation.

### SUMMARY OF THE INVENTION

Provided are systems and methods for real-time sampling and analysis of biomolecules beneath the surface of biological/agricultural tissue/sample. A method for determining fatty acid profiles in agricultural products (for example, seeds) comprises using matrix-assisted laser desorption ionization (MALDI) mass spectroscopy or laser ablation electrospray ionization (LAESI) mass spectroscopy. The MALDI or LAESI mass spectroscopy may be used to profile certain fatty acid traits, such as docosahexanoic acid (DHA), in oil seeds. The disclosed method may be used for a high throughput and/or automated screening of agricultural products, such as seeds, for desirable traits or events.

In one aspect, provided is a system for determining fatty acid profiles in an agricultural sample. The system comprises:
(a) at least one agricultural sample comprising fatty acids;
(b) a laser unit to emit energy at the sample to ablate the sample and generate an ablation plume;
(c) an ionization source to generate a spray plume to intercept the ablation plume and generate ions from fatty acids within the sample; and
(d) a mass spectrometer to detect the ions.

In one embodiment, the emitted energy has a wavelength at an absorption band of one of an OH group, a CH group, a NH group, and a COOH group. In another embodiment, the emitted energy is coupled into the sample by water in the sample.

In one embodiment, the agricultural sample comprises a seed. In a further embodiment, the emitted energy for ablating the sample does not destroy viability or germination of the seed. In another embodiment, the system does not comprise a seed holder or seed container. In another embodiment, pericarp of the seed remains intact and is not ablated. In another embodiment, the system is adapted in a high-throughput format. In another embodiment, the fatty acids comprise at least one of docosahexanoic acid (DHA), linolenic acid, oleic acid, stearidonic acid (SDA), erucic acid, saturated fatty acid. In another embodiment, the seed comprises transgenic seed.

In another aspect, provided is a method of comparing biomolecule profiles of seed tissues using the LAESI-MS methods disclosed. The method comprises:
(a) vaporizing part of a seed tissue with a laser pulse to generate an ablation plume with a laser unit;
(b) generating a spray plume with an ionization source;
(c) intercepting the ablation plume with the spray plume to generate biomolecule ions within the seed tissue;
(d) detecting the biomolecule ions with a mass spectrometer; and
(e) generating biomolecule profiles based on data from the mass spectrometer.

In one embodiment, the laser pulse is generated from a source including a member selected from the group consisting of an infrared (IR) laser, a laser in visible spectrum, and an ultraviolet (UV) laser. In another embodiment, the ionization source is selected from the group consisting of electrospray ionization (ESI), coronal discharge, chemical ionization, thermal emission ionization, fast atom bombardment, photoionization, and inductively coupled plasma (CIP) ionization. In another embodiment, the seed comprises soybean seed or canola seed. In another embodiment, the seed tissue comprises seed coat, hilum, or cotyledon. In another embodiment, the seed tissue is selected from the group consisting of seed coat, hilum, cotyledon, abaxial parenchyma, adaxial parenchyma, vascular bundle, abaxial epidermis, aleurone, parenchyma, housglass, palisade, shoot meristem, plumule, vascular, epidermis, root meristem, and combinations thereof. In another embodiment, the biomolecule profiles relate to colors and/or pigmentation of the seed tissue. In another embodiment, the biomolecule profiles comprise profiles of at least one of triacyl glycerols, diacyl glycerols, flavanoids, small peptides (<5 kDa), small proteins (<20 kDa), and large proteins (>20 kDa). In another embodiment, no seed holder nor seed container is used. The advantages of the LASEI-MS methods provided herein include at least one of (1) non-destructive to maintain viability of the seed; (2) adaptable for high-throughput format and/or automation; and (3) specific seed orientation/position can be achieved to target a particular seed tissue. The laser intensity to vaporize the seed tissue can be adjusted according to different seed tissues, for example, seed coat, hilum, or cotyledon.

In another aspect, provided is a method for determining fatty acid profiles in an agricultural sample. The method comprises:
(a) ablating the sample with a laser pulse to generate an ablation plume with a laser unit;
(b) generating a spray plume with an ionization source;
(c) intercepting the ablation plume with the spray plume to generate ions from fatty acids within the sample; and
(d) detecting the ions with a mass spectrometer.

In one embodiment, the laser pulse is generated from a source including a member selected from the group consisting of an infrared (IR) laser, a laser in visible spectrum, and an ultraviolet (UV) laser. In another embodiment, the ionization source is selected from the group consisting of electrospray ionization (ESI), coronal discharge, low temperature plasma (LTP), chemical ionization, direct analysis in real time (DART), desorption electrospray ionization (DESI), nano-desorption electrospray ionization (nano-DESI), thermal emission ionization, fast atom bombardment, photoionization, and inductively coupled plasma (ICP) ionization.

In one embodiment, the agricultural sample comprises a seed. In a further embodiment, the laser pulse for ablating the sample does not destroy viability or germination of the seed. In another embodiment, no seed holder nor seed container is used. In another embodiment, pericarp of the seed remain intact and is not ablated. In another embodiment, the method is adapted in a high-throughput format. In another embodiment, the fatty acids comprise at least one of docosahexanoic acid (DHA), linolenic acid, oleic acid, stearidonic acid (SDA), erucic acid, saturated fatty acid. In another embodiment, the seed comprises transgenic seed.

In another aspect, provided is a method for measuring fatty acids profile of an agricultural sample. The method comprises:
(a) obtaining ions from fatty acids within the sample by laser ablation and ionization;
(b) detecting the ions with a mass spectrometer; and
(c) comparing the mass spectrum with a known standard.

In another aspect, provided is a method for measuring fatty acids profile of an agricultural sample. The method comprises:
(a) vaporizing surface material at a removal site by pulse of laser;
(b) dissolving molecules of vaporized material in a liquid at a collection site;
(c) providing the liquid containing the dissolved molecules to an ion source to generate ions from fatty acids within the sample; and
(d) detecting the ions with a mass spectrometer.

In one embodiment of the methods provided, the agricultural sample comprises a seed. In a further embodiment, the laser pulse for ablating the sample does not destroy viability or germination of the seed. In another embodiment, no seed holder nor seed container is used. In another embodiment, the removal site does not comprise pericarp of the seed. In another embodiment, the method is adapted in a high-throughput format. In another embodiment, the fatty acids comprise at least one of docosahexanoic acid (DHA), linolenic acid, oleic acid, stearidonic acid (SDA), erucic acid, saturated fatty acid. In another embodiment, the seed comprises transgenic seed.

In another aspect, provided is a method for determining fatty acid profiles in an agricultural product. The method comprises:
(a) extracting oil from the agricultural product;
(b) preparing a matrix-assisted laser desorption ionization (MALDI) sample comprising the extracted oil and a MALDI matrix;
(c) imaging the MALDI sample using a mass analyzer; and
(d) analyzing mass spectral data obtained from the mass analyzer to determine the fatty acid profiles.

In one embodiment, the agricultural sample comprises a seed. In a further embodiment, the laser pulse for ablating the sample does not destroy viability or germination of the seed. In another embodiment, no seed holder nor seed container is used. In another embodiment, the method is adapted in a high-throughput format. In another embodiment, the fatty acids comprise at least one of docosahexanoic acid (DHA), linolenic acid, oleic acid, stearidonic acid (SDA), erucic acid, saturated fatty acid. In another embodiment, the seed comprises transgenic seed. In another embodiment, the mass analyzer includes a member selected from the group consisting of mass spectrometer (MS), time-of-flight mass spectrometer (TOF MS), ion-trap mass spectrometer, and a quadrupole mass spectrometer (QTOF MS). In another embodiment, the seed includes a member selected from the group consisting of soybean, corn, canola, rapeseed, sunflower, peanut, safflower, palm, cotton, wheat, maize, soybean, rice, alfalfa, oat, apple seed, Arabidopsis thaliana, banana, barley, bean, linseed, melon, olive, pea, pepper, poplar, broccoli, castor bean, citrus, clover, coconut, coffee, maize, strawberry, sugarbeet, sugarcane, sweetgum, tea, tobacco, tomato, rye, sorghum, cucumber, Douglas fir, Eucalyptus, Loblolly pine, Radiata pine, Southern pine, and turf.

In one embodiment, extracting oil from the agricultural product includes extracting the oil with a solvent. In a further embodiment, the solvent includes a member selected from the group consisting of hexane, decane, petroleum ether, alcohol, toluene, benzene, tetrahydrofuran, dimethyl sulfoxide, trimethylsulfonium hydroxide, acetonitrile, methylene chloride, and combinations thereof. In another embodiment, the MALDI matrix includes a member selected from the group consisting of 2,5-dihydroxybenzoic acid (DHB), 3,5-dimethoxy-4-hydroxycinnamic acid (SA), α-cyano-4-hydroxycinnamic acid (CHCA), 4-hydroxy-3-methoxycinnamic acid, picolinic acid, 3-hydroxy picolinic acid, and combinations thereof.

In one embodiment, the oil is extracted from the agricultural product using a push pin device. The seed is pierced using a sharp pointy device with a pointy tip, such as “push pin” to extract or obtain a thin layer of oil or any liquid within the agricultural product. In a further or alternative embodiment, the extracted oil is eluted from the push pin device with a solvent comprising methanol. In a further or alternative embodiment, the extracted oil is eluted from the push pin device with a solvent comprising methanol. In a further embodiment, the extracted oil is eluted from the push pin with a solvent comprising chloroform and methanol. In a further embodiment, ratio of chloroform and methanol is from 10:1 to 1:10. In a further embodiment, ratio of chloroform and methanol is from 4:1 to 2:1.

In another aspect, provided is a method for determining fatty acid profiles in an agricultural product. The method comprises:
(a) depositing at least one seed sample to surface of a glass slide having double sided adhesive tape thereon;
(b) extracting oil from the at least one seed sample;
(c) transferring the extracted oil to a clean glass slide;
(d) preparing a matrix-assisted laser desorption ionization (MALDI) sample comprising the extracted oil and a MALDI matrix;
(e) imaging the MALDI sample using a mass analyzer; and
(f) analyzing mass spectral data obtained from the mass analyzer to determine the fatty acid profiles.

In one embodiment, the agricultural sample comprises a seed. In a further embodiment, the laser pulse for ablating the sample does not destroy viability or germination of the seed. In another embodiment, no seed holder nor seed container is used. In another embodiment, the method is adapted in a high-throughput format. In another embodiment, the fatty acids comprise at least one of docosahexanoic acid (DHA), linolenic acid, oleic acid, stearidonic acid (SDA), erucic acid, saturated fatty acid. In another embodiment, the seed comprises transgenic seed.

In another embodiment, the “push pin” device can be modified using selectively adsorbing or absorbing materials on the surface, such as C18 modified silica resin. In another embodiment, the ‘push pin” device with extracted oils is directly used to perform mass spectrometric analysis using a DART or DESI source.

### BRIEF DESCRIPTION OF THE DRAWINGS

FIG. 1 shows an exemplary embodiment of the disclosed method where canola seed samples are prepared and analyzed using MALDI mass spectrometry (MALDI-TOF MS) technique.

FIG. 2 shows exemplary mass spectra for determining fatty acid profiles in, for example, transgenic canola seeds.

FIGS. 3A and 3B show exemplary embodiments of the disclosed systems and/or methods where canola seed samples are prepared and analyzed using LAESI mass spectrometry (LEASI-MS) technique.

FIGS. 4A-4D show exemplary mass spectra of fatty acid profiles from samples with known compositions. These exemplary mass spectra can serve as positive controls in subsequent experiments.

FIG. 5A shows an exemplary MS profile of a sample from canola seed showing triacyl glycerol peak at 907.778. This peak is subject to MS/MS analysis for showing its signature fingerprint as shown in FIG. 5B.

FIGS. 6A and 6B show a similar analysis using a push pin method to extract oil from seed followed by MALDI analysis.

FIGS. 7A and 7B show exemplary MS/MS of ions from extracted oil from seed. Peaks around 350.154 indicate ions from DHA.

FIGS. 8A and 8B show additional analysis of various samples using similar to approaches used in FIGS. 5 and 6, where different seeds are used and different extraction methods are used.

FIGS. 9A-9C, 10A-10B, and 11A-11C shows additional analysis of various samples using similar to approaches used in FIGS. 5, 6 and 8, where positive controls (similar to FIG. 4) are labeled with blue dots.

### DETAILED DESCRIPTION OF THE INVENTION

The invention relates to sampling biological tissues, including seeds, using LASER ablation electrospray ionization technique, and analysis of the material directly using mass spectrometry. The systems and methods provided can be used to ablate surfaces of biological tissues to characterize or profile biomolecules on or beneath the surface to correlate with the desired trait. The systems and methods provided also can be modified to be a high throughput method for event selection.

Non-destructive analysis of seeds or similar tissues to determine their oil content is important for the selection events with desired traits, while keeping them viable. Though techniques for sampling, such as chipping or extraction exist, they offer limited high throughput capability. Sample tissues (for example, seeds) can be analyzed using MALDI-TOF/TOF. Laser ablation instruments can perform sampling of surface molecules or beneath surface molecules using an IR laser to ablate the surface (A) and vaporize the analytes, which may be detected using mass spectrometry(B). The systems and methods provided for sampling biomolecules in a continuous fashion from living tissues, can aid in the determination of traits or related characteristics to select a specific event. Sampling can be done to determine the complete lipid profile, or a specific lipid based on their parent masses and fragmentation characteristics. Similarly the systems and methods provided can also be leveraged to determine the relative amounts of different lipids present in the sample. The systems and methods provided also allow for minimal sampling of the seeds wherein no actual tissue is isolated for the analysis, rather the material is suspended as a plume and drawn into the inlet of a mass spectrometer for further analysis. The systems and methods provided can be applied to other biomolecules, such as flavanoids, peptides, and other metabolites, which can be used for event or trait characterization. The systems and methods provided can be used with most mass spectrometers available in the market.

The systems and methods provided enable at least one of the following advantages:
(1) monitoring and/or characterizing the change in fatty acid content of triacyl glycerols present in the seeds of events that have either been genetically modified or generated by other breeding means;
(2) monitoring and/or characterizing the change in amount of flavoids or isoflavanoids;
(3) monitoring and/or characterizing the composition of the seed coats to determine/correlate their physical properties with the event/trait;
(4) selecting or rejecting/screening events in a high throughput format; and
(5) monitoring and/or characterizing pesticides and their metabolites on the surface of leaves as a measure of bioavailability and/or application efficacy

The production of transgenic plants has become routine for many plant species, but the current methodologies are labor intensive and unpredictable. Thus, a goal of the methods and systems disclosed is to provide a sampling method suitable for high-throughput applications in a consistent and/or concise manner.

The present disclosures now will be described more fully hereinafter, but not all embodiments of the disclosure are shown. While the disclosure has been described with reference to certain embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof.

In some embodiments, a method for determining fatty acid profiles in agricultural products, such as seeds, includes extracting oil from the agricultural products, preparing a matrix-assisted laser desorption ionization (MALDI) sample comprising the extracted oil and a MALDI matrix, and imaging the MALDI sample using a mass analyzer.

In other embodiments, a method for determining fatty acid profiles in agricultural products, such as seeds, includes ablating the agricultural products with an optical beam (e.g., pulses of laser light) to generate an ablation plume, ionizing the ablation plume, directing the ionized ablation plume into an inlet of a mass analyzer, and performing mass analysis on the ionized ablation plume.

In certain embodiments, a method for high throughput screening of seeds includes preparing a matrix-assisted laser desorption ionization MALDI sample that comprises an oil extracted from the seeds and a MALDI matrix or preparing an ionized plume sample of ablated seeds, and imaging the sample using a mass analyzer.

As used herein, the phrase “oils” refer to any liquid material within the agricultural samples at room temperature. An oil may include either a hydrocarbon oil (i.e., an oil whose molecule contains only atoms of carbon and hydrogen) or a non-hydrocarbon oil (i.e., an oil whose molecule contains at least at least one atom that is neither carbon nor hydrogen).

Hydrocarbon oils may include, for example, straight, branched, or cyclic alkane compounds with 6 or more carbon atoms. Some hydrocarbon oils, for example, have one or more carbon-carbon double bond, one or more carbon-carbon triple bond, or one or more aromatic ring, possibly in combination with each other and/or in combination with one or more alkane group.

As used herein, the phrase “vector” refers to a piece of DNA, typically double-stranded, which can have inserted into it a piece of foreign DNA. The vector can be, for example, of plasmid or viral origin, which typically encodes a selectable or screenable marker or transgenes. The vector is used to transport the foreign or heterologous DNA into a suitable host cell. Once in the host cell, the vector can replicate independently of or coincidental with the host chromosomal DNA. Alternatively, the vector can target insertion of the foreign or heterologous DNA into a host chromosome.

As used herein, the phrase “transgene vector” refers to a vector that contains an inserted segment of DNA, the “transgene” that is transcribed into mRNA or replicated as a RNA within a host cell. The phrase “transgene” refers not only to that portion of inserted DNA that is converted into RNA, but also those portions of the vector that are necessary for the transcription or replication of the RNA. A transgene typically comprises a gene-of-interest but needs not necessarily comprise a polynucleotide sequence that contains an open reading frame capable of producing a protein.

As used herein, the phrase “transformed” or “transformation” refers to the introduction of DNA into a cell. The phrases “transformant” or “transgenic” refers to plant cells, plants, and the like that have been transformed or have undergone a transformation procedure. The introduced DNA is usually in the form of a vector containing an inserted piece of DNA.

As used herein, the phrase “selectable marker” or “selectable marker gene” refers to a gene that is optionally used in plant transformation to, for example, protect the plant cells from a selective agent or provide resistance/tolerance to a selective agent. Only those cells or plants that receive a functional selectable marker are capable of dividing or growing under conditions having a selective agent. Examples of selective agents can include, for example, antibiotics, including spectinomycin, neomycin, kanamycin, paromomycin, gentamicin, and hygromycin. These selectable markers include gene for neomycin phosphotransferase (npt II), which expresses an enzyme conferring resistance to the antibiotic kanamycin, and genes for the related antibiotics neomycin, paromomycin, gentamicin, and G418, or the gene for hygromycin phosphotransferase (hpt), which expresses an enzyme conferring resistance to hygromycin. Other selectable marker genes can include genes encoding herbicide resistance including Bar (resistance against BASTA® (glufosinate ammonium), or phosphinothricin (PPT)), acetolactate synthase (ALS, resistance against inhibitors, such as sulfonylureas (SUs), imidazolinones (IMIs), triazolopyrimidines (TPs), pyrimidinyl oxybenzoates (POBs), and sulfonylamino carbonyl triazolinones that prevent the first step in the synthesis of the branched-chain amino acids), glyphosate, 2,4-D, and metal resistance or sensitivity. The phrase “marker-positive” refers to plants that have been transformed to include the selectable marker gene.

Various selectable or detectable markers can be incorporated into the chosen expression vector to allow identification and selection of transformed plants, or transformants. Many methods are available to confirm the expression of selection markers in transformed plants, including, for example, DNA sequencing and PCR (polymerase chain reaction), Southern blotting, RNA blotting, immunological methods for detection of a protein expressed from the vector, e.g., precipitated protein that mediates phosphinothricin resistance, or other proteins, such as reporter genes β-glucuronidase (GUS), luciferase, green fluorescent protein (GFP), DsRed, β-galactosidase, chloramphenicol acetyltransferase (CAT), alkaline phosphatase, and the like (See Sambrook, et al., Molecular Cloning: A Laboratory Manual, Third Edition, Cold Spring Harbor Press, N.Y., 2001, the content of which is incorporated herein by reference in its entirety).

Selectable marker genes are utilized for the selection of transformed cells or tissues. Selectable marker genes include genes encoding antibiotic resistance, such as those encoding neomycin phosphotransferase II (NEO) and hygromycin phosphotransferase (HPT) as well as genes conferring resistance to herbicidal compounds. Herbicide resistance genes generally code for a modified target protein insensitive to the herbicide or for an enzyme that degrades or detoxifies the herbicide in the plant before it can act. See DeBlock et al. (1987) EMBO J., 6:2513-2518; DeBlock et al. (1989) Plant Physiol., 91:691-704; Fromm et al. (1990) 8:833-839; Gordon-Kamm et al. (1990) 2:603-618). For example, resistance to glyphosate or sulfonylurea herbicides has been obtained by using genes coding for the mutant target enzymes, 5-enolpyruvylshikimate-3-phosphate synthase (EPSPS) and acetolactate synthase (ALS). Resistance to glufosinate ammonium, bromoxynil, and 2,4-dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides. Enzymes/genes for 2,4-D resistance have been previously disclosed in US 2009/0093366 and WO 2007/053482, the contents of which are hereby incorporated by reference in their entireties.

Other herbicides can inhibit the growing point or meristem, including imidazolinone or sulfonylurea. Exemplary genes in this category code for mutant ALS and AHAS enzyme as described, for example, by Lee et al., EMBO J. 7:1241 (1988); and Miki et al., Theon. Appl. Genet. 80:449 (1990), respectively.

Glyphosate resistance genes include mutant 5-enolpyruvylshikimate-3-phosphate synthase (EPSPs) genes (via the introduction of recombinant nucleic acids and/or various forms of in vivo mutagenesis of native EPSPs genes), aroA genes and glyphosate acetyl transferase (GAT) genes, respectively). Resistance genes for other phosphono compounds include glufosinate (phosphinothricin acetyl transferase (PAT) genes from Streptomyces species, including Streptomyces hygroscopicus and Streptomyces viridichromogenes), and pyridinoxy or phenoxy proprionic acids and cyclohexones (ACCase inhibitor-encoding genes), See, for example, U.S. Pat. No. 4,940,835 to Shah, et al. and U.S. Pat. No. 6,248,876 to Barry et al., which disclose nucleotide sequences of forms of EPSPs which can confer glyphosate resistance to a plant. A DNA molecule encoding a mutant aroA gene can be obtained under ATCC accession number 39256, and the nucleotide sequence of the mutant gene is disclosed in U.S. Pat. No. 4,769,061 to Comai, European patent application No. 0 333 033 to Kumada et al., and U.S. Pat. No. 4,975,374 to Goodman et al., disclosing nucleotide sequences of glutamine synthetase genes which confer resistance to herbicides, such as L-phosphinothricin. The nucleotide sequence of a PAT gene is provided in European application No. 0 242 246 to Leemans et al. Also DeGreef et al., Bio/Technology 7:61 (1989), describes the production of transgenic plants that express chimeric bar genes coding for PAT activity. Exemplary of genes conferring resistance to phenoxy proprionic acids and cyclohexones, including sethoxydim and haloxyfop, are the Accl-S1, Accl-S2 and Accl-S3 genes described by Marshall et al., Theon. Appl. Genet. 83:435 (1992). GAT genes capable of conferring glyphosate resistance are described in WO 2005012515 to Castle et al. Genes conferring resistance to 2,4-D, fop and pyridyloxy auxin herbicides are described in WO 2005107437 and U.S. patent application Ser. No. 11/587,893.

Other herbicides can inhibit photosynthesis, including triazine (psbA and 1s+ genes) or benzonitrile (nitrilase gene). Przibila et al., Plant Cell 3:169 (1991), describes the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442. Cloning and expression of DNA coding for a glutathione S-transferase is described by Hayes et al., Biochem. J. 285:173 (1992).

For purposes of the present invention, selectable marker genes include, but are not limited to genes encoding: neomycin phosphotransferase II (Fraley et al. (1986) CRC Critical Reviews in Plant Science, 4:1-25); cyanamide hydratase (Maier-Greiner et al. (1991) Proc. Natl. Acad. Sci. USA, 88:4250-4264); aspartate kinase; dihydrodipicolinate synthase (Perl et al. (1993) Bio/Technology, 11:715-718); tryptophan decarboxylase (Goddijn et al. (1993) Plant Mol. Bio., 22:907-912); dihydrodipicolinate synthase and desensitized aspartade kinase (Perl et al. (1993) Bio/Technology, 11:715-718); bar gene (Toki et al. (1992) Plant Physiol., 100:1503-1507 and Meagher et al. (1996) and Crop Sci., 36:1367); tryptophan decarboxylase (Goddijn et al. (1993) Plant Mol. Biol., 22:907-912); neomycin phosphotransferase (NEO) (Southern et al. (1982) J. Mol. Appl. Gen., 1:327; hygromycin phosphotransferase (HPT or HYG) (Shimizu et al. (1986) Mol. Cell Biol., 6:1074); dihydrofolate reductase (DHFR) (Kwok et al. (1986) PNAS USA 4552); phosphinothricin acetyltransferase (DeBlock et al. (1987) EMBO J., 6:2513); 2,2-dichloropropionic acid dehalogenase (Buchanan-Wollatron et al. (1989) J. Cell. Biochem. 13D:330); acetohydroxyacid synthase (Anderson et al., U.S. Pat. No. 4,761,373; Haughn et al. (1988) Mol. Gen. Genet. 221:266); 5-enolpyruvyl-shikimate-phosphate synthase (aroA) (Comai et al. (1985) Nature 317:741); haloarylnitrilase (Stalker et al., published PCT application WO87/04181); acetyl-coenzyme A carboxylase (Parker et al. (1990) Plant Physiol. 92:1220); dihydropteroate synthase (sul I) (Guerineau et al. (1990) Plant Mol. Biol. 15:127); and 32 kD photosystem II polypeptide (psbA) (Hirschberg et al. (1983) Science, 222:1346).

Also included are genes encoding resistance to: chloramphenicol (Herrera-Estrella et al. (1983) EMBO J., 2:987-992); methotrexate (Herrera-Estrella et al. (1983) Nature, 303:209-213; Meijer et al. (1991) Plant Mol Bio., 16:807-820 (1991); hygromycin (Waldron et al. (1985) Plant Mol. Biol., 5:103-108; Zhijian et al. (1995) Plant Science, 108:219-227 and Meijer et al. (1991) Plant Mol. Bio. 16:807-820); streptomycin (Jones et al. (1987) Mol. Gen. Genet., 210:86-91); spectinomycin (Bretagne-Sagnard et al. (1996) Transgenic Res., 5:131-137); bleomycin (Hille et al. (1986) Plant Mol. Biol., 7:171-176); sulfonamide (Guerineau et al. (1990) Plant Mol. Bio., 15:127-136); bromoxynil (Stalker et al. (1988) Science, 242:419-423); 2,4-D (Streber et al. (1989) Bio/Technology, 7:811-816); glyphosate (Shaw et al. (1986) Science, 233:478-481); and phosphinothricin (DeBlock et al. (1987) EMBO J., 6:2513-2518). All references recited in the disclosure are hereby incorporated by reference in their entireties unless stated otherwise.

The above list of selectable marker and reporter genes are not meant to be limiting. Any reporter or selectable marker gene are encompassed by the present invention. If necessary, such genes can be sequenced by methods known in the art.

The reporter and selectable marker genes are synthesized for optimal expression in the plant. That is, the coding sequence of the gene has been modified to enhance expression in plants. The synthetic marker gene is designed to be expressed in plants at a higher level resulting in higher transformation efficiency. Methods for synthetic optimization of genes are available in the art. In fact, several genes have been optimized to increase expression of the gene product in plants.

The marker gene sequence can be optimized for expression in a particular plant species or alternatively can be modified for optimal expression in plant families. The plant preferred codons may be determined from the codons of highest frequency in the proteins expressed in the largest amount in the particular plant species of interest. See, for example, EPA 0359472; EPA 0385962; WO 91/16432; Perlak et al. (1991) Proc. Natl. Acad. Sci. USA, 88:3324-3328; and Murray et al. (1989) Nucleic Acids Research, 17: 477-498; U.S. Pat. No. 5,380,831; and U.S. Pat. No. 5,436,391, herein incorporated by reference. In this manner, the nucleotide sequences can be optimized for expression in any plant. It is recognized that all or any part of the gene sequence may be optimized or synthetic. That is, fully optimized or partially optimized sequences may also be used.

In addition, several transformation strategies utilizing the Agrobacterium-mediated transformation system have been developed. For example, the binary vector strategy is based on a two-plasmid system where T-DNA is in a different plasmid from the rest of the Ti plasmid. In a co-integration strategy, a small portion of the T-DNA is placed in the same vector as the foreign gene, which vector subsequently recombines with the Ti plasmid.

As used herein, the phrase “explant” refers to a removed section of living tissue or organ from one or more tissues or organs of a subject.

As used herein, the phrase “plant” includes dicotyledons plants and monocotyledons plants. Examples of dicotyledons plants include tobacco, Arabidopsis, soybean, tomato, papaya, canola, sunflower, cotton, alfalfa, potato, grapevine, pigeon pea, pea, Brassica, chickpea, sugar beet, rapeseed, watermelon, melon, pepper, peanut, pumpkin, radish, spinach, squash, broccoli, cabbage, carrot, cauliflower, celery, Chinese cabbage, cucumber, eggplant, and lettuce. Examples of monocotyledons plants include corn, rice, wheat, sugarcane, barley, rye, sorghum, orchids, bamboo, banana, cattails, lilies, oat, onion, millet, and triticale.

Particular embodiments of the disclosed method for determining fatty acid profiles in agricultural products may include using matrix-assisted laser desorption ionization (MALDI), mass spectroscopy, or laser ablation electrospray ionization (LAESI) mass spectroscopy to analyze fatty acids in the agricultural products.

The analyzed agricultural products may include seeds; plant tissues, such as leaf, flower, root, petal; or other agricultural products.

In one particular embodiment, a method for screening seeds to determine their fatty acid profiles may include using matrix-assisted laser desorption ionization (MALDI) mass spectroscopy or laser ablation electrospray ionization (LAESI) mass spectroscopy to analyze fatty acids in the seeds.

The disclosed methods may be used to screen various types of seeds including, but are not limited to, soybean, corn, canola, rapeseed, sunflower, peanut, safflower, palm, cotton, wheat, maize, soybean, rice, alfalfa, oat, apple seed, Arabidopsis thaliana, banana, barley, bean, linseed, melon, olive, pea, pepper, poplar, broccoli, castor bean, citrus, clover, coconut, coffee, maize, strawberry, sugarbeet, sugarcane, sweetgum, tea, tobacco, tomato, lye, sorghum, cucumber, Douglas fir, Eucalyptus, Loblolly pine, Radiata pine, Southern pine, and turf.

A variety of fatty acids may be identified using the disclosed methods including, but are not limited to, docosahexanoic acid (DHA), linolenic acid, oleic acid, stearidonic acid (SDA), erucic acid, saturated fatty acid, and combinations thereof.

Various seed processing, mass spectrum, and laser ablation systems have been described in U.S. Pat. Nos. 8,110,794 and 7,910,881, as well as patent applications US 2012/0025069, US 2011/0294700, US 2008/0274234, and US 2008/0308722, the content of which are incorporated by references in their entireties.

The disclosed methods are mass sensitive, thereby allowing for both qualitative and quantitative analysis of fatty acid profiles in the agricultural products.

Various mass spectrometries may be used for the disclosed methods in combination with MALDI or LAESI techniques. Examples of the mass spectrometries may include, but are not limited to, time of flight mass spectrometer (TOF MS), quadrupole time-of-flight mass spectrometry (QTOF MS), electrospray ionization mass spectrometry (ESI-MS), electrospray mass spectrometry (ES-MS), direct analysis in real time (DART), desorption electrospray ionization (DESI), sector mass spectrometry, ion-trap mass spectrometry, or desorption atmospheric pressure chemical ionization (DAPCI).

When electrospray ionization-based mass spectroscopy system (e.g., ESI-MS, ES-MS, DART, DESI, and nano-DESI) is used, the predominant ions are ammonium, sodium or other adduct ions of triacylglyceride lipids presented in the seeds. These ions may be further broken apart in the mass spectroscopy environment by using more energy (e.g., MS/MS or tandem MS process) to generate fragmentation ions wherein the triacylglyceride lipids loss one or more fatty acid side chains. Based on the parent mass and the fragment mass formed during MS/MS or tandem MS process, the identity of fatty acids in the triacylglyceride lipids of the seeds may be determined. The electrospray ionization-based mass spectroscopy method is an indirect process and requires two steps to determine the fatty acid compositions.

When MALDI mass spectroscopy is used, the predominant ions are sodium ions of triacylglyceride lipids presented in the seeds. Fragmentation of sodiated triacylglyceride compounds in the MALDI-TOF MS mass spectrum shows unique daughter ions corresponding to the sodium salt of polyunsaturated fatty acid (PUFA Na⁺). This feature is in direct contrast to the ions formed in the electrospray ionization-based mass spectroscopy system. This unique feature of MALDI-TOF MS mass spectroscopy allows for a fast screening of seed samples containing a specific polyunsaturated fatty acid, such as DHA, without specific isolation of the precursor ions.

MALDI-TOF MS analyses of the triacylglyceride lipids produce fragment ions that are diagnostic for the presence of particular fatty acids in the triacylglyceride lipids. On the other hand, the electrospray ionization-based MS (ESI-MS) analyses produce ammonium or other adduct ions which under MS/MS conditions generate neutral losses that may be used indirectly to identify the presence of specific fatty acids in the triacylglyceride lipids.

When desired, the selected substance ions may be isolated and then fragmented prior to mass analysis in order, for example, to obtain higher identification accuracy.

Regardless of mass spectroscopy techniques, it is possible to assign mass spectrometric signals (i.e., peaks) to specific fatty acids present in the seed sample based on the fragmentation patterns in the mass spectrum. The mass spectrometry may be optimized to scan through a particular mass range (such as at m/z of about 900-1400) and look for a set of specified product ions and/or neutral loss ions in the MS/MS scans. These product ions and/or neutral loss ions are signatures of specific polyunsaturated fatty acids (PUFA) and may be used for screening seeds for PUFA content, both qualitative and quantitatively.

In one particular embodiment of present disclosure, a method for determining fatty acid profiles in agricultural products may include using laser ablation electrospray ionization technique (LAESI) in combination of mass spectroscopy to analyze the agricultural products. The ablated surfaces of seeds may be analyzed to characterize or profile biomolecules on or beneath the seed surface to correlate with the desired traits or events. Furthermore, the method may be modified to enable a high throughput screening for event selection.

In some embodiments, the disclosed method for determining fatty acid profiles in agricultural product may include ablating the agricultural product with an optical beam to generate an ablation plume, ionizing the ablation plume, directing the ionized ablation plume into an inlet of a mass analyzer, performing mass analysis on the ionized ablation plume, and analyzing mass spectral data obtained from the mass analysis to determine fatty acid profiles.

In one embodiment, the disclosed method is used to determine fatty acid profiles in seeds. The method may include ablating the seeds with an optical beam (e.g., pluses of laser light) to generate an ablation plume, ionizing the ablation plume, directing the ionized ablation plume into an inlet of a mass analyzer, performing mass analysis on the ionized ablation plume, and analyzing mass spectral data obtained from the mass analysis to determine fatty acid profiles in the seeds.

The disclosed methods of screening and analyzing seeds may be used for individual seed or batches of seeds. Furthermore, the disclosed methods may allow for high throughput sorting of oil seeds based on their polyunsaturated fatty acid (PUFA) content.

The disclosed method may be used to determine the oil content in seeds with a higher sensitivity and lower detection limit than conventional methods. Particularly, the disclosed method may allow for non-destructive, high throughput analysis of DHA fatty acid in oil seeds.

Additionally, the disclosed method may be used for screening and testing of seeds at grain elevators, oil processing plants, food formulations laboratories and the like, or in seed breeding applications where large numbers of small samples must be analyzed to make immediate planting decisions so that time and resources are not wasted in growing plants without desirable traits.

In addition to oil and fatty acid contents, the disclosed method may be used for analysis of protein content (such as crude protein), amino acid content (e.g., Asp, Tyr, Phe, Lys, Met, Cys, Trp, Thr), sugar content, gluten content, ash content, lipid content, carbohydrate content, starch content, or combinations thereof (e.g., oil and protein content). Furthermore, the disclosed method may be used to determine the contents of oligosaccharide, alpha-amylase and/or isoflavone in agricultural products. For example, the disclosed method may be used to identify and determine isoflavone content in seeds. Isoflavones are known to enhance human health and animal health (e.g., pigs, poultry, fish, and cows).

The disclosed method of analyzing agricultural products may be used for various applications. Non-limiting examples of such applications may include monitoring the change in fatty acid content of triacylglycerides present in the seeds of events that have either been genetically modified or generated by other breeding means; monitoring the change in flavonoids or isoflavanoids content present in the seeds of certain events, such as genetically modification or other breeding means; monitoring the composition of the seed coats to correlate their physical properties with certain event or trait, electing or rejecting events based on fatty acid content, flavonoids or isoflavanoids content, or composition of the seed coats; and monitoring pesticides and their metabolites on the surface of leaves as a measure of bioavailability and/or application efficacy.

Accordingly, the disclosed methods may be used for quality assurance (QA) and quality control (QC) by assuring that unwanted fatty acid composition characteristics are identified prior to a grain handler making purchasing or processing decisions, or a seed breeder making planting decisions.

### EXAMPLES

### Example 1

### Systems and Methods Provided in High-Throughput Format

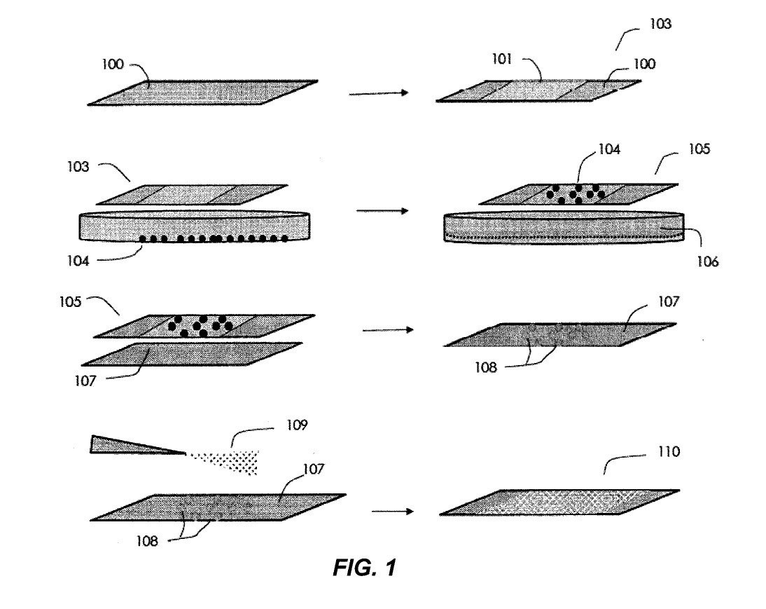

FIG. 1 shows one embodiment of the disclosed method for screening seeds in a high throughput manner using MALDI-TOF MS/MS system. A double sided adhesive tape 101 is placed on one surface of a glass slide 100. The resulting glass slide 103 is then held over seeds 104 and gentle pressed against seeds 104 to provide glass slide 105 having seeds 104 of similar size adhered to it by adhesive tape 101. Glass slide 105 having seed 104 is slightly immersed in a solvent reservoir 106 and quickly placed on a clean glass slide 107 to extracted oil 108 in the seeds 104. Then, several layers of MALDI matrix 109 are applied onto the glass slide 107 having extracted oil 108 in 5-10 minute intervals to produce sample slide 110. The MALDI-TOF MS/MS is used to image sample slide 110. Mass spectral data from each pixel of the MALDI-mass spectrometric image is used to extract peaks arising from specific fatty acids. The obtained fatty acid profiles may be used to match with the seeds and/or to sort events. The selected seeds may then be planted and grown.

Any suitable solvent known in the art for extracting oil from seeds may be used. Examples of such solvents may include, but not limited to, hexane, decane, petroleum ether, alcohol, toluene, benzene, tetrahydrofuran, dimethyl sulfoxide, trimethylsulfonium hydroxide, acetonitrile, or methylene chloride. The amount of solvent used depends on the amount of sample analyzed. The volume of solvent sufficient to extract a detectable amount of oil and the method of extraction are known in the art.

Various known MALDI matrix may be used including, but are not limited to, 2,5-dihydroxybenzoic acid (DHB), 3,5-dimethoxy-4-hydroxycinnamic acid (SA), α-cyano-4-hydroxycinnamic acid (CHCA), 4-hydroxy-3-methoxycinnamic acid, picolinic acid, 3-hydroxy picolinic acid, and combinations thereof.

The disclosed method using MALDI-TOF MS/MS technique may provide a high throughput means for screening and analyzing certain characteristics of seeds. When desired, the disclosed method may be adapted for automation.

### Example 2

### Oil from Canola Seed Tested with Systems and Methods Provided

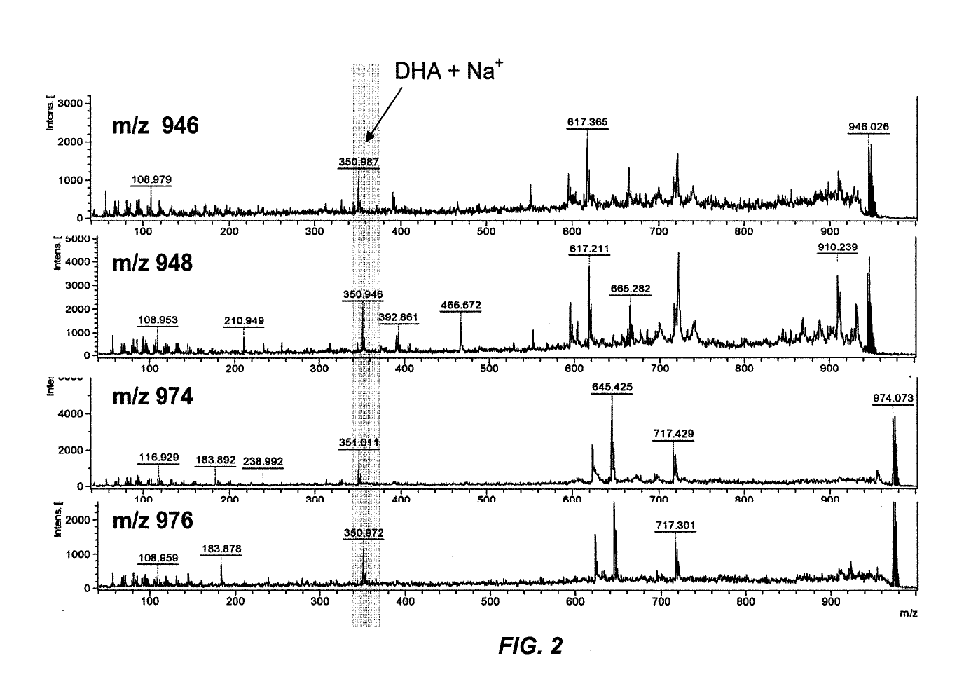

Oil can be extracted from canola seeds and prepared for analysis using a MALDI system provided. In some embodiments, the canola seeds are from transgenic plants having enhanced or modified oil profiles as compared to their non-transgenic parents. FIG. 2 shows mass spectra of the MALDI-TOF MS fragmentation data obtained from four different precursor ions (m/z of 946, 948, 974 and 978) of DHA oil available from Martek Biosciences Corporation. The mass spectral data show the presence of DHA Na⁺ signature ion at m/z of about 350 regardless of the precursor ions.

In one particular embodiment of present disclosure, a method for analyzing seeds may include extracting oil from the seeds, preparing a matrix-assisted laser desorption ionization (MALDI) sample comprising the extracted oil and a MALDI matrix, and imaging the MALDI sample using a MALDI-TOF MS or MALDI MS mass analyzer. The resulting MALDI-TOF MS/MS image may be used to map the regions containing specific fatty acids back to the specific seeds and the event may be probed further.

The disclosed methods using MALDI-TOF MS/MS system may be adapted for screening agricultural products, such as seeds, for certain traits or events in a high throughput manner. Furthermore, a continuous analysis in an automated fashion may be achieved. MALDI-TOF MS/MS system operates at very low pressure environments and may be designed to encompass several seeds in one analysis. For example, in some embodiment of the disclosed methods, hundreds of seeds may be screened in one analysis.

MALDI-TOF MS/MS involves laser pulses focused on a small sample plate (MALDI sample) comprising analyte molecules embedded in a low molecular weight, UV-absorbing matrix (MALDI matrix) that enhances ionization of the analyte molecules. The MALDI matrix facilitates intact desorption and ionization of the analyte molecules.

In one embodiment, the disclosed method may include extracting oil from at least one seed, depositing a matrix-assisted laser desorption ionization (MALDI) matrix on the extracted oil to produce a MALDI sample, imaging the MALDI sample using a mass analyzer, and analyzing mass spectral data obtained from the mass analyzer to determine fatty acid profiles in the seed.

### Example 3

### Systems and Methods Provided in High-Throughput Format

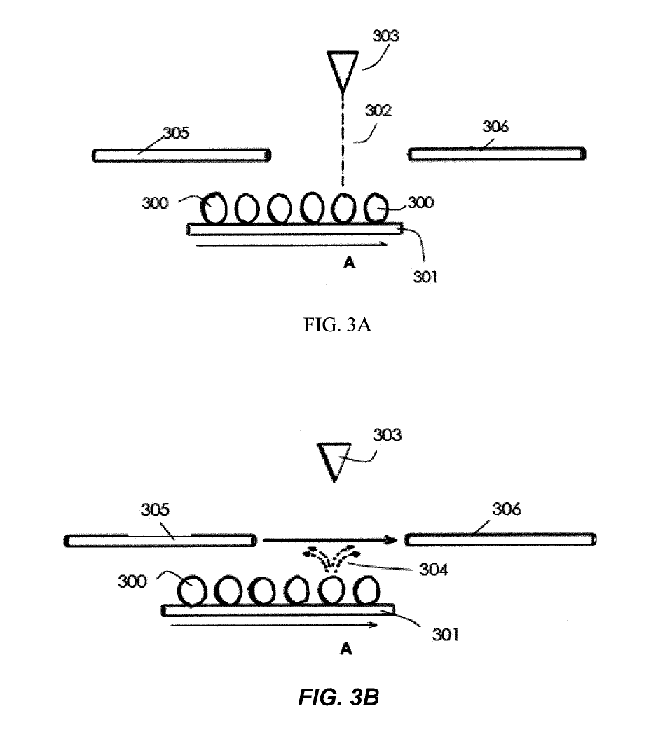

FIG. 3 illustrates a non-limiting example of the disclosed method for screening seeds in a high throughput manner using LAESI-MS technique. Seeds 300 are placed on a moving stage 301 moving in direction of A. Seeds 300 are ablated with pulses of laser light 302 generated from a laser light source 303 (FIG. 3A), causing an ejection of ablation plume 304. As shown in FIG. 3B, the ablation plume 304 is then ionized with an ion source 305, and the ionized ablation plume was directed into an inlet of a mass analysis device 306 (mass spectroscopy) for mass analysis. The laser ablation may be performed on the seed sample using LAESI DP-1000 available from Protea Biosciences Group Incorporation.

Many laser light sources may be used to ablate the seeds including, but are not limited to, an infrared laser, a laser in the visible spectrum, or an ultraviolet (UV) laser.

Laser ablation only removes neutral molecules on the surface of seeds being analyzed. Therefore, the neutral molecules must be ionized prior to analysis using a mass analyzer. Any conventional ionization mechanism may be used including, but are not limited to, electrospray ionization, coronal discharge, chemical ionization, thermal emission ionization, fast atom bombardment, photoionization, inductively coupled plasma ionization, and other plasma based methods of ionization, such as direct analysis in real time (DART).

Various types of mass analyzers may be used in combination with LAESI technique. Examples of such mass analyzers may include, but not limited to, the mass analyzer with RF ion traps, ion cyclotron resonance, time-of-flight measuring devices, quadrupole filters, magnetic sector fields, or the like.

Unlike conventional fatty acid methyl ester (FAME) analysis, the disclosed method using LAESI-MS technique offers a non-destructive analysis of seeds. Therefore, the disclosed method using LEASI-MS technique may be used to determine the fatty acid profiles of seeds in a breeding program. The disclosed method may allow for improved breeding programs, wherein non-destructive seed sampling may be conducted while maintaining the identity of individuals from the seed sampler to the field. As a result, a high throughput breeding program may be achieved, wherein a population of seeds having desired fatty acid profiles is more effectively bulked in a shorter period of time and with less field and labor resources required.

Furthermore, the disclosed method using LAESI-MS technique allows for minimal sampling of the seeds wherein no actual seed tissue is isolated for the analysis. Instead, the seed tissue is suspended as a plume and drawn into the inlet of a mass spectrometer for further analysis.

### Example 4

### MS Profile and MS/MS Signature Fingerprint

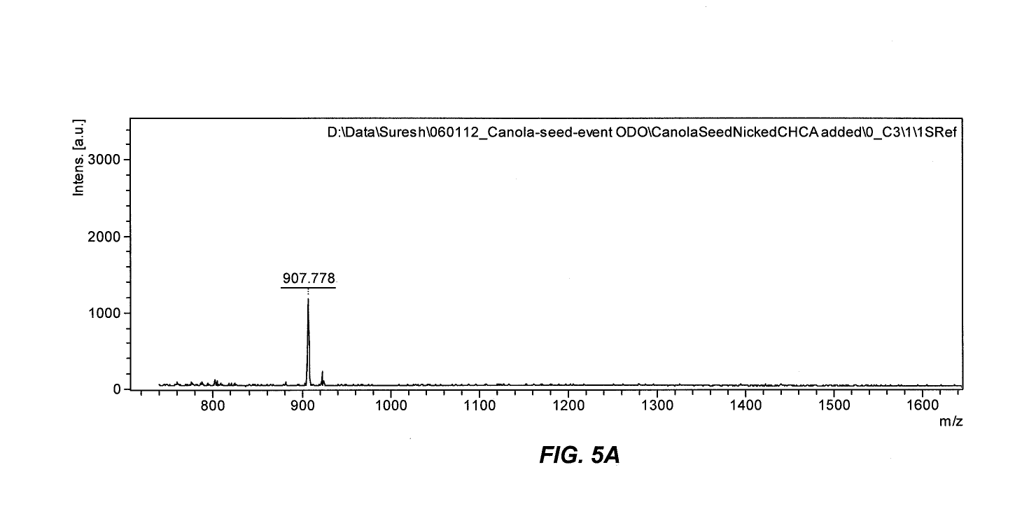

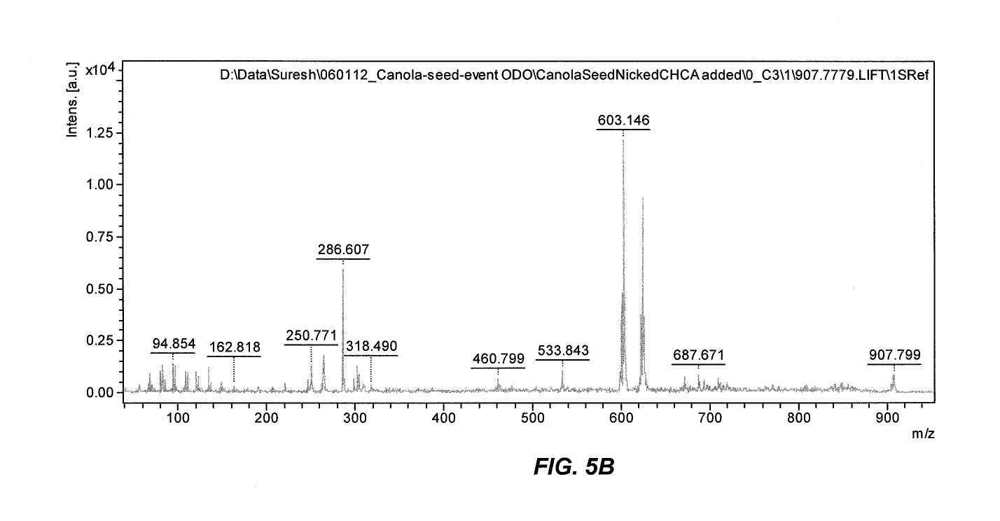

One objective is to obtain oil sample from seed without destroying seed viability. MALDI analysis shows presence of fragments in the m/z 350 indicating presence of DHA+Na+ ions. Canola seeds are used and a small piece of seed is nicked to be placed on MALDI target with 1 μL CHCA matrix. LIFT spectra of m/z 90 can be obtained and fragments 603, 625 belong to C18:2—TAG; 302 is sodiated C18:2. An exemplary MS profile of a sample from canola seed showing olive acid peak at 907.778 is shown in FIG. 5A. This olive acid peak is subject to MS/MS analysis for showing its signature fingerprint as shown in FIG. 5B.

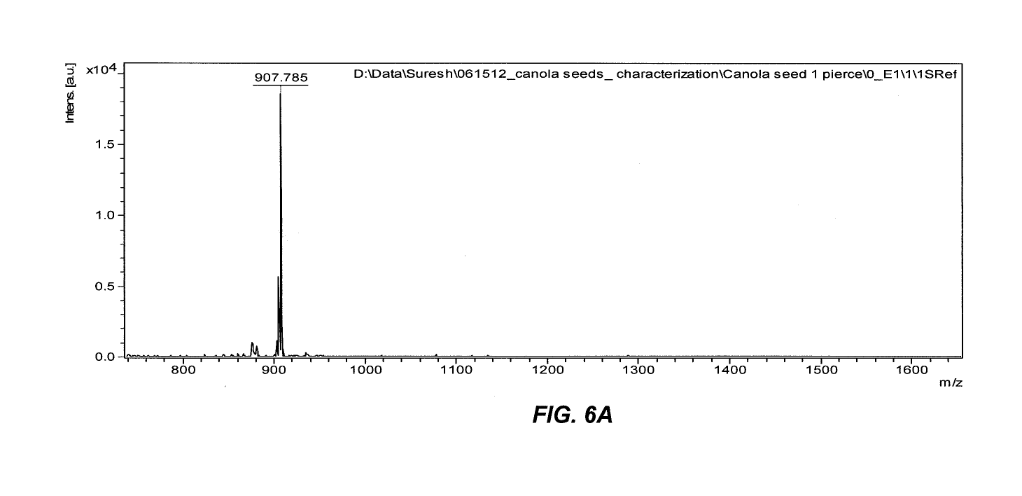

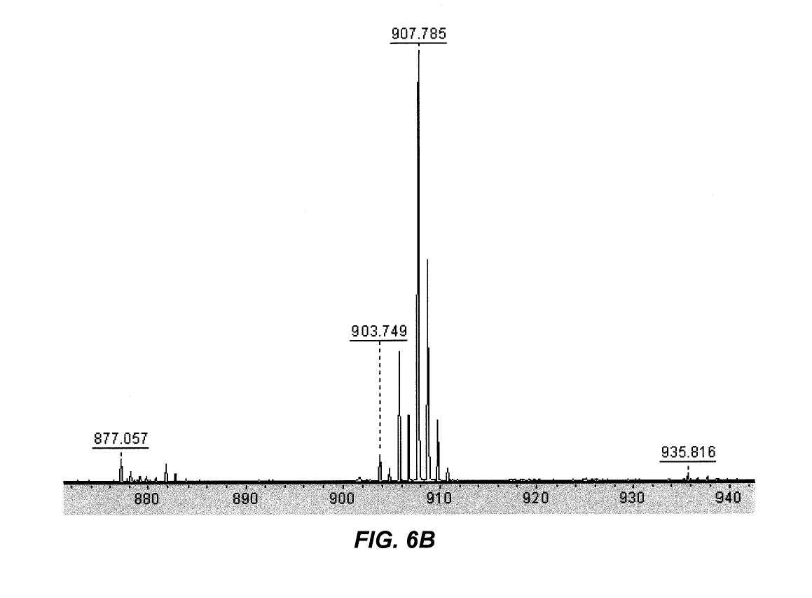

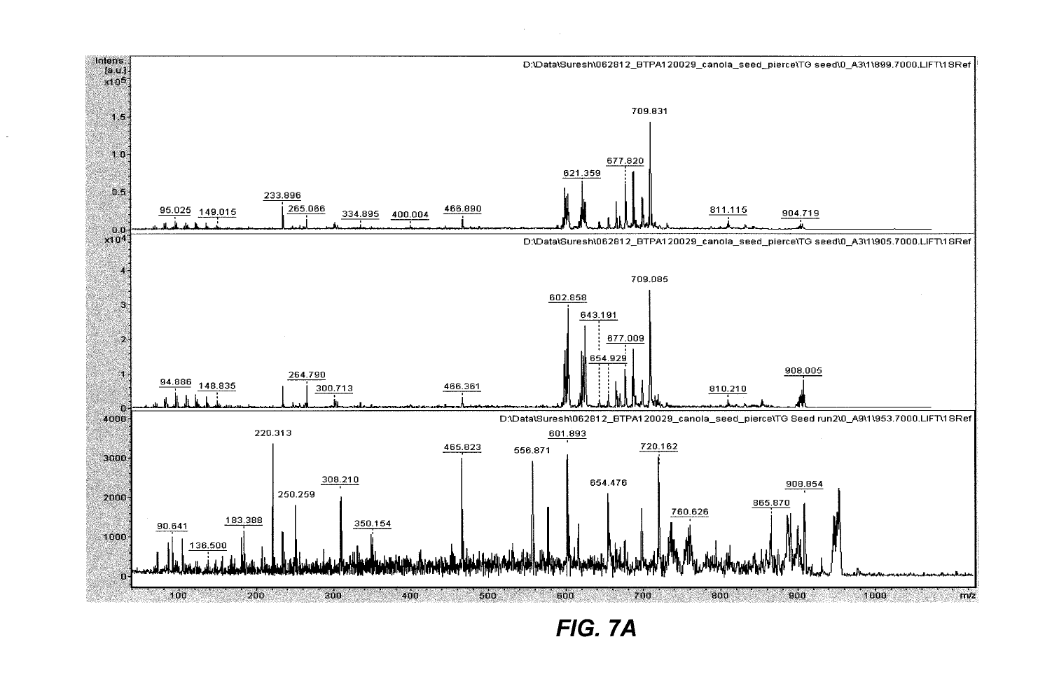

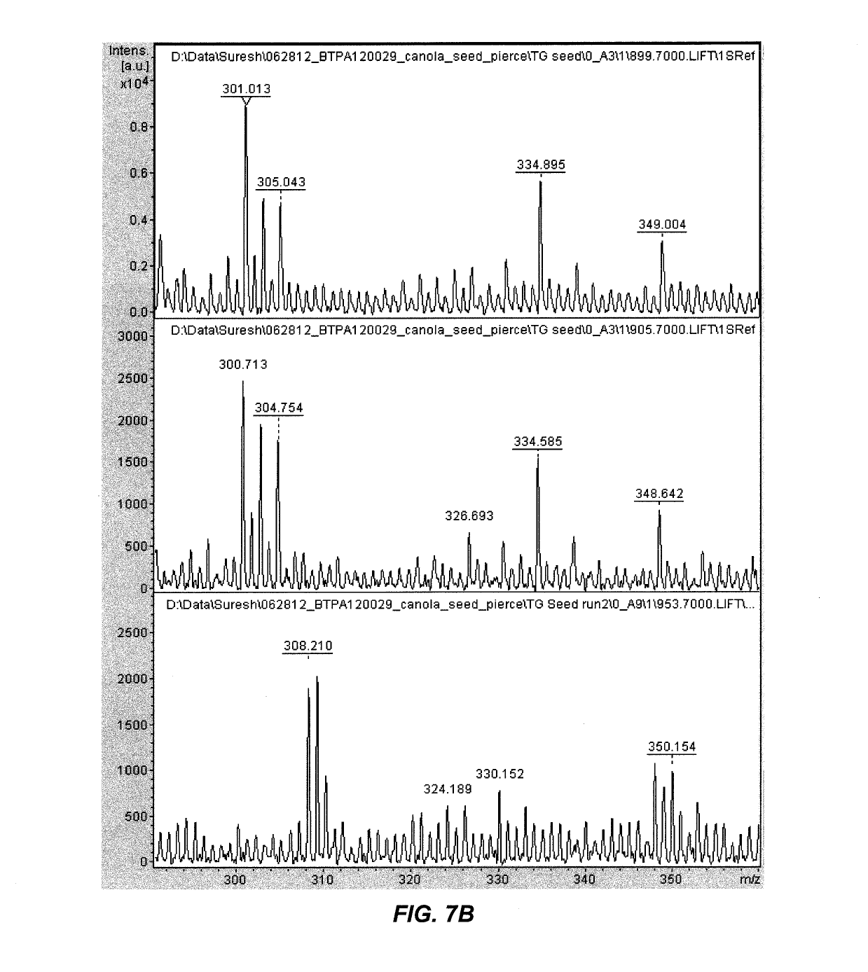

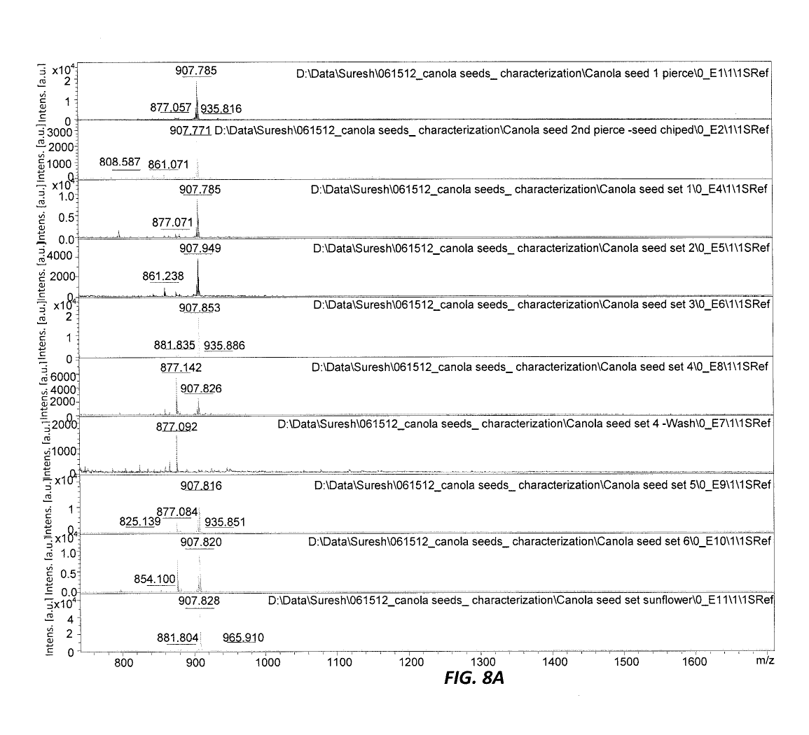

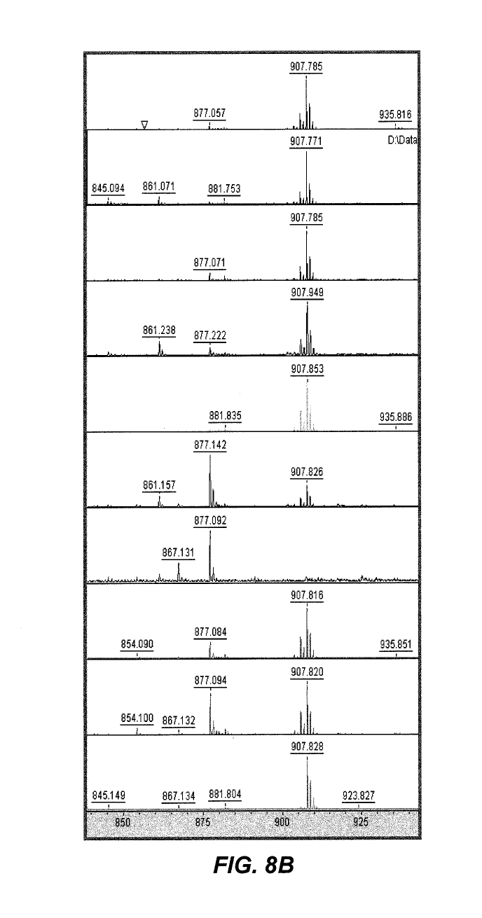

In addition, a push pin is used to prick the seeds to a depth of about 2 mm and the tip of the push pin is rinsed using 0.5 μL of chloroform:methanol (3:1) solution to elude the extracted oil for mass spec analysis. 0.5 μL of CHCA solution is used for matrix crystals. Spots on the MALDI target are shown in FIGS. 6A and 6B as similar to FIGS. 5A and 5B. Spectra of additional samples are shown in FIGS. 8A and 8B, where different seeds are used and different extraction methods are used. FIGS. 7A and 7B show exemplary MS/MS of ions from extracted oil from seed (seed pierce). Peaks around 350.154 indicate ions from DHA.

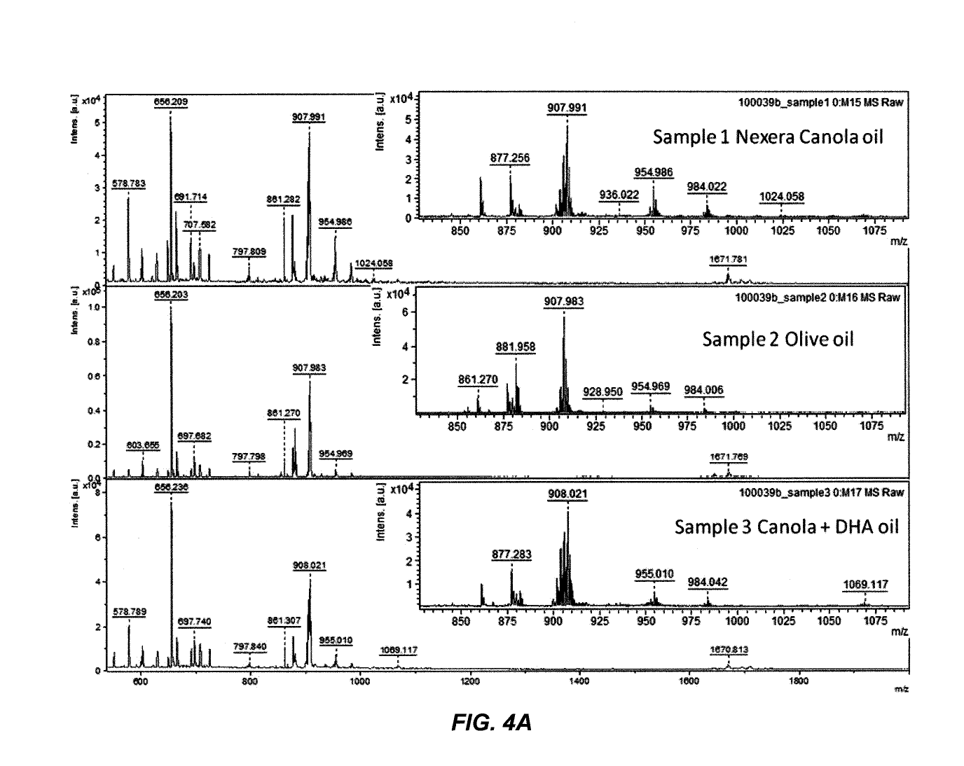

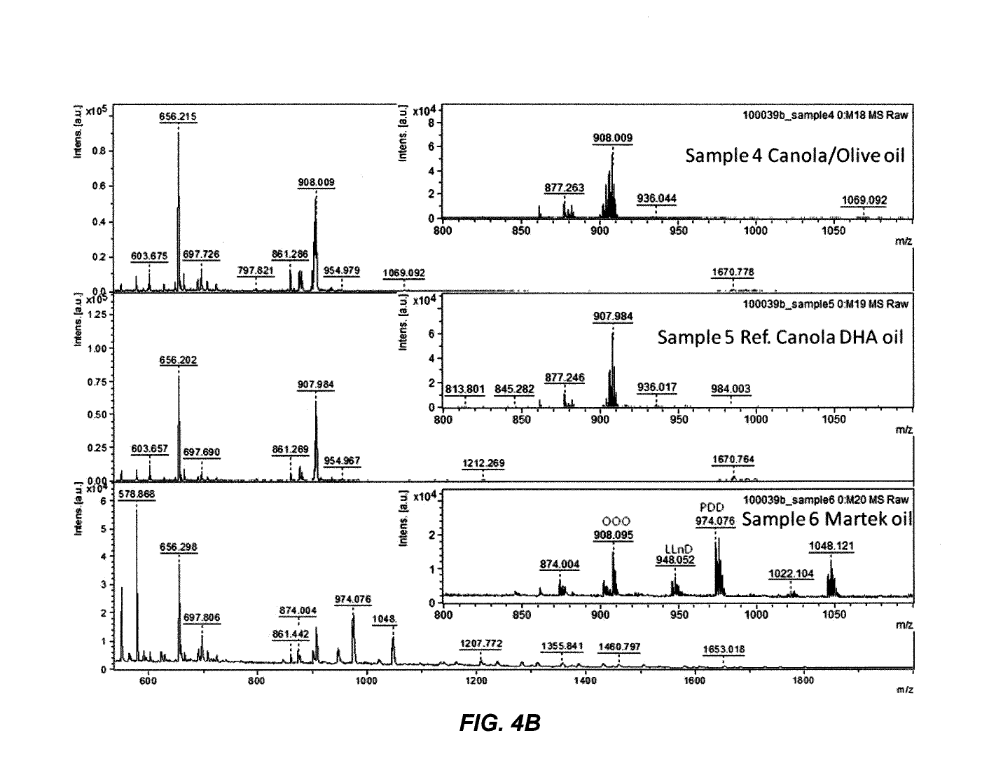

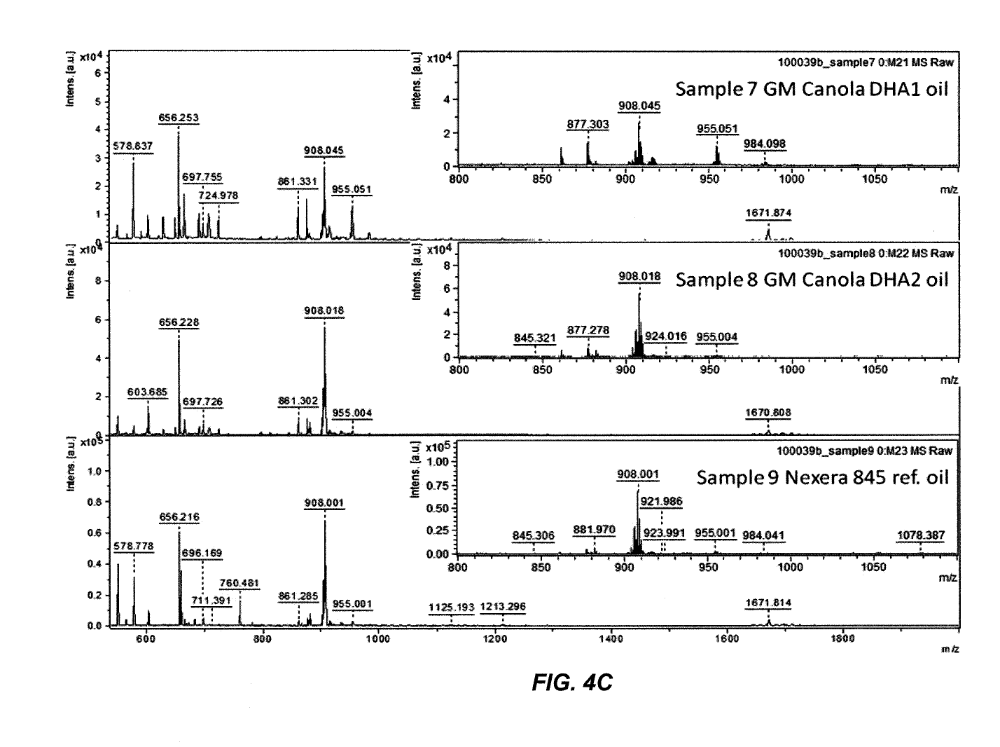

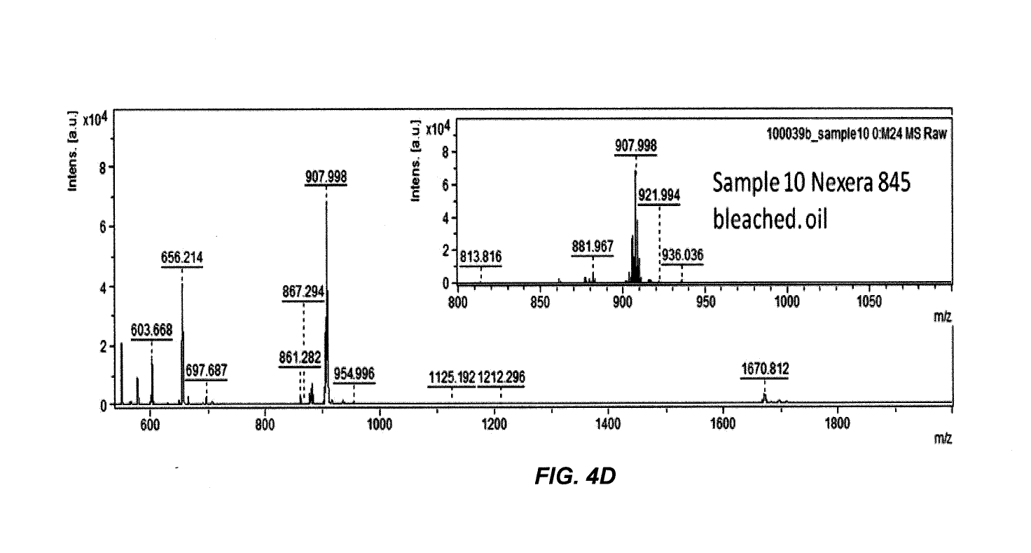

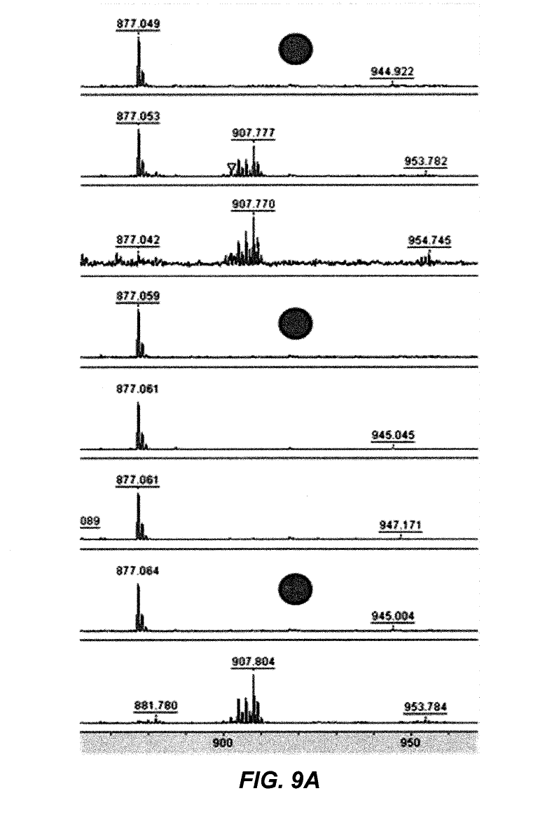

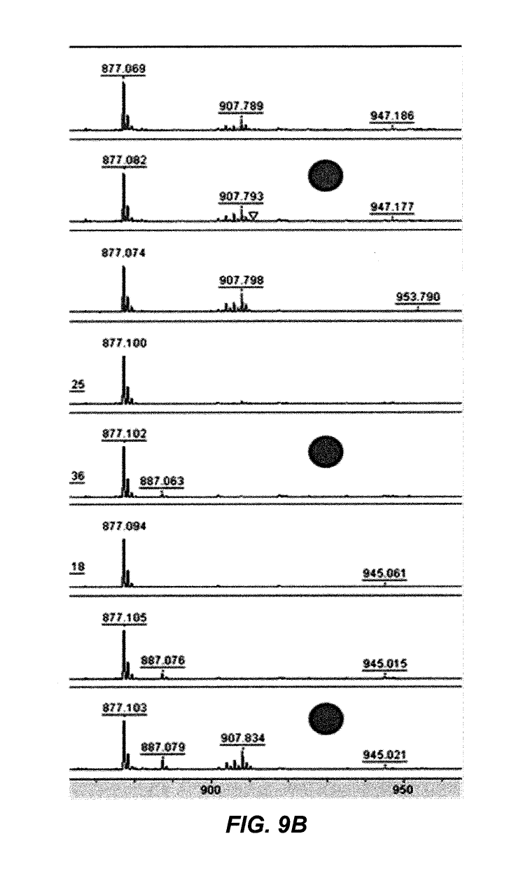

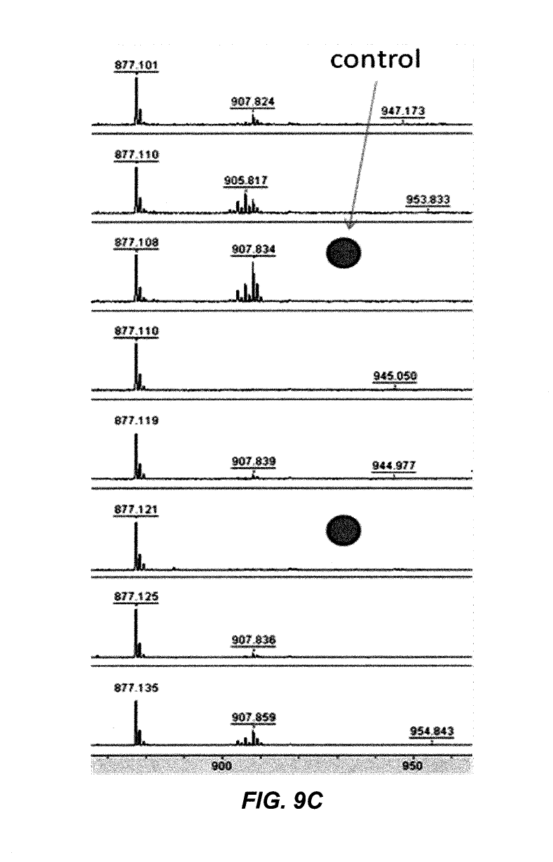

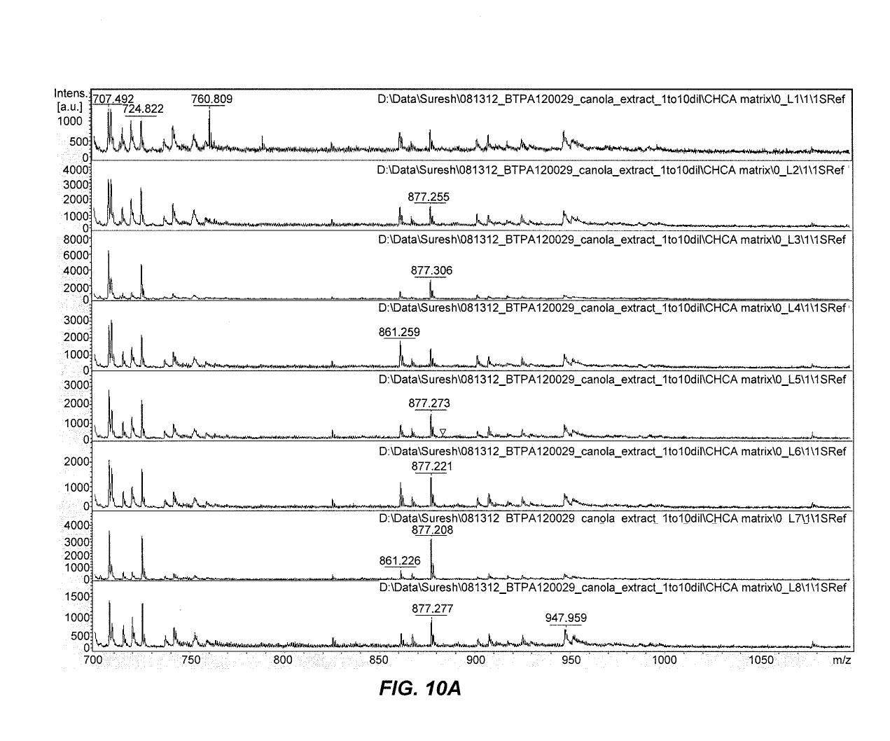

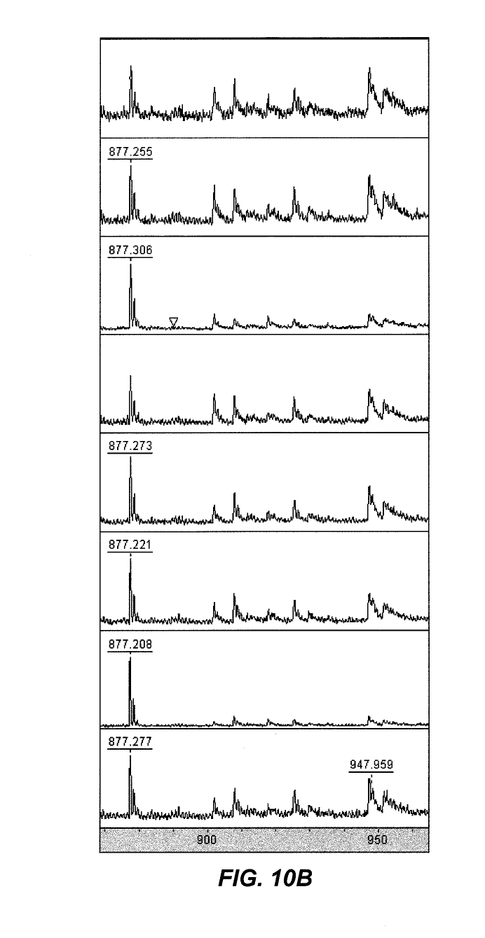

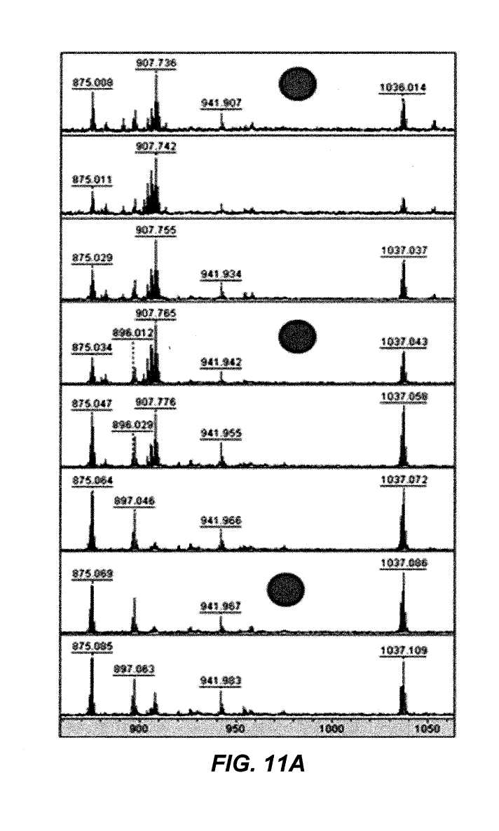

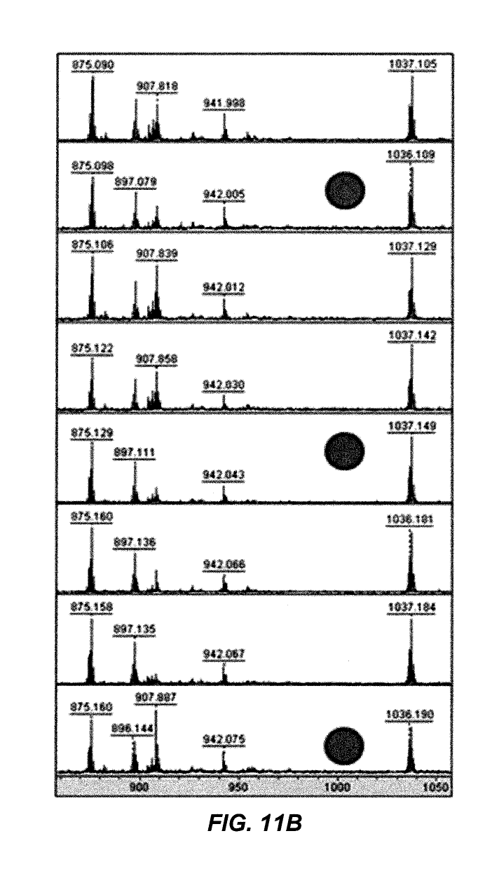

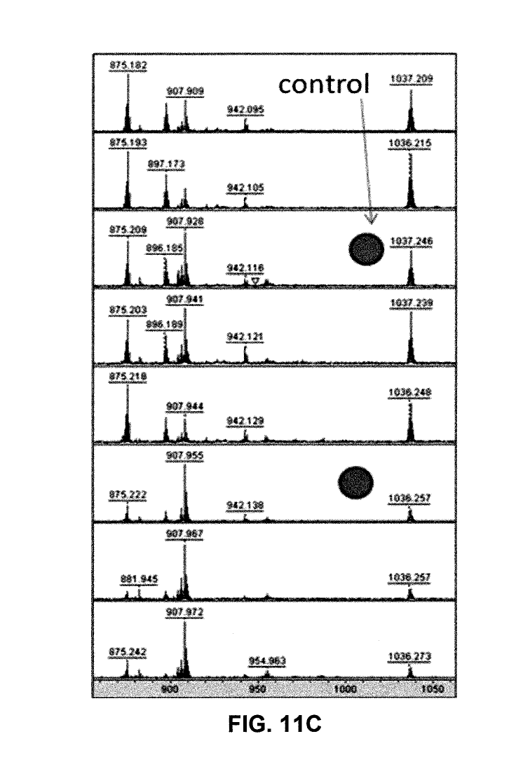

Seeds from transgenic plants along with their controls are subject to MALDI-MS analysis to determine their TAG profiles. All the seeds are pierced with a push pin and eluted onto a plant and analyzed similarly as procedures set for the in this example. Data are correlated with FAME analysis. FIGS. 9A-9C, 10A-10B, and 11A-11C shows additional analysis of various samples using similar to approaches used in FIGS. 5, 6 and 8, where positive controls (similar to FIG. 4) are labeled with blue dots.

While the disclosure has been described by reference to various specific embodiments, it should be understood that numerous changes may be made within the spirit and scope of the inventive concepts described. Accordingly, it is intended that the invention not be limited to the described embodiments, but will have full scope defined by the language of the following claims.

What is claimed is:

1. A system for determining fatty acid profiles in an agricultural sample, comprising, at least one agricultural sample comprising fatty acids;
a laser unit to emit energy at the sample to ablate the sample and generate an ablation plume;
an ionization source to generate a spray plume to intercept the ablation plume and generate ions from fatty acids within the sample; and
a mass spectrometer to detect the ions.

2. The system of claim 1, wherein the emitted energy has a wavelength at an absorption band of one of an OH group, a CH group, a NH group, and a COOH group.

3. The system of claim 1, wherein the emitted energy is coupled into the sample by water in the sample.

4. The system of claim 1, wherein the agricultural sample comprises a seed.

5. The system of claim 4, wherein the emitted energy for ablating the sample does not destroy viability or germination of the seed.

6. The system of claim 4, wherein the system does not comprise a seed holder or seed container.

7. The system of claim 4, wherein pericarp of the seed remains intact and is not ablated.

8. The system of claim 1, wherein the system is adapted in a high-throughput format.

9. The system of claim 1, wherein the fatty acids comprise at least one of docosahexanoic acid (DHA), linolenic acid, oleic acid, stearidonic acid (SDA), erucic acid, saturated fatty acid.

10. A method of comparing biomolecule profiles of seed tissues, comprising, vaporizing part of a seed tissue with a laser pulse to generate an ablation plume with a laser unit;
generating a spray plume with an ionization source;
intercepting the ablation plume with the spray plume to generate biomolecule ions within the seed tissue;
detecting the biomolecule ions with a mass spectrometer; and
generating biomolecule profiles based on data from the mass spectrometer.

11. The method of claim 10, wherein the laser pulse is generated from a source including a member selected from the group consisting of an infrared (IR) laser, a laser in visible spectrum, and an ultraviolet (UV) laser.

12. The method of claim 10, wherein the ionization source is selected from the group consisting of electrospray ionization (ESI), coronal discharge, chemical ionization, thermal emission ionization, fast atom bombardment, photoionization, and inductively coupled plasma (CIP) ionization.

13. The method of claim 10, wherein the seed comprises soybean seed or canola seed.

14. The method of claim 10, wherein the seed tissue comprises seed coat, hilum, or cotyledon.

15. The method of claim 10, wherein the laser pulse does not destroy viability or generation of the seed.

16. The method of claim 10, wherein no seed holder nor seed container is used.

17. The method of claim 10, wherein the biomolecule profiles comprise profiles of at least one of triacyl glycerols, diacyl glycerols, flavanoids, small peptides (<5 kDa), small proteins (<20 kDa), and large proteins (>20 kDa).

18. The method of claim 10, wherein the method is adapted in a high-throughput format.

19. The method of claim 10, wherein the orientation or position of the seed is arranged to target a particular seed tissue.

20. A method for measuring fatty acids profile of an agricultural sample, comprising, vaporizing surface material at a removal site by pulse of laser;
dissolving molecules of vaporized material in a liquid at an collection site;
providing the liquid containing the dissolved molecules to an ion source to generate ions from fatty acids within the sample; and
detecting the ions with a mass spectrometer.

21. The method of claim 20, wherein the agricultural sample comprises a seed.

22. The method of claim 21, wherein the laser pulse for ablating the sample does not destroy viability or germination of the seed.

23. The method of claim 21, wherein no seed holder nor seed container is used.

24. The method of claim 20, wherein the removal site does not comprise pericarp of the seed.

25. The method of claim 20, wherein the method is adapted in a high-throughput format.

26. The method of claim 20, wherein the fatty acids comprise at least one of docosahexanoic acid (DHA), linolenic acid, oleic acid, stearidonic acid (SDA), erucic acid, saturated fatty acid.
